(19) United States
(12) Patent Application Publication
(10) Pub. No.: US 2002/0018748 A1
(43) Pub. Date: Feb. 14, 2002

(54) Method for targeted radiotherapy, pain palliation and imaging of osteoblastic skeletal tumors and composition therefore

(76) Inventors: Stanley Satz, Surfside, FL (US); Roseanne Satz, Sunny Isles Beach, FL (US); Alfredo Cuaron, Hollywood, FL (US)

Correspondence Address: CLIFFORD G. FRAYNE, Suite 7A, 136 Drum Point Road, Brick, NJ, 08723, US

(21) Appl. No.: 09/825,492

(22) Filed: Apr. 3, 2001

Related U.S. Application Data
(60) Provisional application No. 60/194,119, filed on Apr. 3, 2000.

(51) Int. Cl. A61K 51/00

(52) U.S. Cl. 424/1.11

(57) ABSTRACT

A method for treating bone metastases and like skeletal tumors in an individual which comprises treating said individual with a dose of a physiologically acceptable amount.

### RELATED APPLICATIONS

This application claims the benefit of the priority date of provisional application 60/194,119 filed Apr. 3, 2000.

### BACKGROUND OF THE INVENTION

1. Field of the Invention

The present invention relates to biochemically-targeted radiotherapy, and more particularly to an improved method for detecting and treating bone metastases and other skeletal tumors.

2. Description of the Prior Art

Most malignant tumors have the capacity to form new foci of disease (metastases) in skeleton and other organs by distributing clusters of malignant cells through circulating blood or by lymphatic pathways. Once established in bone, malignant cells divide and grow, progressing to bone destruction and remodeling. Bone metastases usually show a mixed pattern of bone destruction and increased bone formation, although the destructive process is predominant. Most bone metastases associated with prostate carcinoma are almost exclusively osteoblastic (bone forming). Lesions associated with breast carcinoma and other cancers can be exclusively osteoblastic or osteolytic (bone destructing), but most commonly exhibit mixed characteristics. Most metastases occur in bones with a high proportion of red marrow, with the most common sites in the axial skeleton. Bone metastases are found in 85% of terminal patients with breast, prostate, or lung cancers, as well as in other malignancies, including renal (25%), rectal (13%), pancreatic (13%), thyroid (12%), stomach (11%), colon and ovary (10% each).

Once cancer has spread through metastases there is no curative option. The only alternative is to palliate the intense and unrelenting pain caused by tumor growth in bone to improve the quality of the patient's remaining life. Radiotherapy and surgery offer ways of reducing the tumor mass in specific regions of the body that are accessible through surgical techniques or by high doses of radiation. Neither technique is applicable to the destruction of widely disseminated metastases. Chemotherapy, although in widespread use, has proved to be of limited effectiveness in treating most cancer types. This is because of the low therapeutic index (the ratio of the cytotoxic drug absorbed dose received by the target cells as compared to that of normal tissues) of anti-cancer drugs, as well as to the intrinsic or acquired drug resistance that often characterizes tumor cells. The low therapeutic index of cytotoxic agents is also the cause of the severe side effects associated with chemotherapy.

Radiopharmaceutical agents have been tried employing beta emitting bone-seeking radionuclides, such as ³²P-ortho-phosphate, ¹⁸⁶Re-hydroxy-ethylene-diphosphonate (HEDP), ¹⁵³Sm-ethylene-diamine tetramethylene-phosphonic acid (EDTMP), and ¹¹⁷Sn^{m}-diethylene-triamine-penta-acetic acid (DTPA). Such agents are incorporated into a calcium phosphate component of hydroxyapatite crystals in mineral bone. Radioisotopes of radium and other alkaline-earth metals, such as strontium, exchange with calcium within the crystalline lattice and remain there for a long period because of the low turnover rate of calcium in mature bone mineral. The regional bone uptake of all of these radiopharmaceuticals is proportional to the regional bone forming activity and shows selective uptake by bone metastases in greater proportion than by normal bone as well as a rapid clearance from soft tissue and normal bone.

All the aforementioned bone-seeking radionuclides have shown to be effective in palliating pain in bone metastases with varying degrees of side effects and levels of efficacy. The Food and Drug Administration (FDA) has only approved ⁸⁹Sr chloride and ¹⁵³Sm-EDTMP for pain palliation application. A survey made in the United States in 1995 showed that ⁸⁹Sr had been used in 99% of patients treated with bone-seeking radionuclides, with an overall effective pain palliation of 81%, and with 15% achieving a complete remission of pain.

According to the United States Pharmacopoeia (USP), ⁸⁹Sr chloride is supplied as a sterile, non-pyrogenic, aqueous solution with radioactive concentration of 1 mCi (37 MBq)/ml and specific activity of 80-167 μCi (2.96-6.17 MBq)/mg of strontium. It is administered intravenously as a single injection. Patients may experience facial flushing during the injection, but administering the dose slowly over at least two minutes can reduce this effect. Since ⁸⁹Sr has almost no gamma emission, it may be safely administered in outpatient setting without hazard to family and personnel.

There is no general consensus on the optimal dose of ⁸⁹Sr-chloride to be administered for pain palliation. In Canada, the usual dose is 10.8 mCi (400 MBq). The Procedure Guideline for Bone Pain Treatment of the Society of Nuclear Medicine suggests activities in the range of 40-60 μCi (1.48-2.22 MBq)/kg but the approved dose by the Federal Drug Administration (FDA) is 4 mCi (148 MBq). In a survey made by the American Society for Therapeutic Radiology and Oncology the preferences are divided between two well-defined dose levels. The most common is 4 mCi (148 MBq), administered in 73% of cases, while 10.8 mCi (400 MBq) was given to 27% of patients.

Duration of response after ⁸⁹Sr administration ranges from 3 to 12 months, with a mean of 6 months. It has been reported that patients with limited skeletal metastases respond better than those with more widespread tumors. Investigators using the ¹⁸⁶Re- and ¹⁵³Sm- diphosphonate compounds have reported similar results, but the response may not be as long as with longer lived ⁸⁹Sr. Patients may receive repeat doses of strontium-89 at 3-month intervals. Additional injections produce responses comparable with those seen initially and with no major toxicity.

Although the ratio of the uptake of ⁸⁹Sr chloride by metastases as compared to the uptake in normal bones is higher than 10 to 1, the toxic effects of bone-seeking radionuclides is related to bone marrow irradiation by the radioactive atoms concentrated in neighboring bone, either in the metastases or in normal bone. The relative contribution of radioactivity in the metastasis and in the normal bone to the toxic effects is not known, but toxic effects are more noticeable in patients with widespread disease. Reduction in the count of thrombocytes and leukocytes in blood is the main toxic effect, as well as a decrease in platelet counts of between 30% and 70% of baseline. However, patients only occasionally meet criteria for toxicity as defined by assessment of cytotoxic chemotherapy. The time of maximum depression in the platelet count usually occurs between four and eight weeks after injection and blood count values tend to return to previous levels, with partial to complete recovery within 6 months.

Palliative doses of bone-seeking radionuclides could be effective in reducing tumor mass and periosteal stretching by growing metastases as well as the stimulation of nerve endings by substances released by the destructive process, but the activity of the dose is not sufficient to destroy the tumors. For effective treatment, these doses need to be greatly increased to deliver a destructive radiation dose to the metastases. Unfortunately, in doing so, the uptake of the radionuclide by normal bone would also be incremented and the toxic effects on bone marrow would be further exacerbated.

The clinical experience gained with ⁸⁹Sr chloride has been obtained with a low specific activity product (80-167 μCi [2.96-617 MBq]/mg of strontium) because the only available method of production of the radionuclide was based on slow-neutron irradiation of highly enriched stable strontium-88 in a thermal nuclear reactor by the reaction ⁸⁸Sr(n, y), according to the equation:

⁸⁸Sr+n→⁸⁹Sr+Y

The resulting product from this reaction contains atoms of both the target (non-radioactive ⁸⁸Sr) and the product (radioactive ⁸⁹Sr), both with the same atomic number, which prevents their separation by chemical means. Therefore, the product is necessarily of a relatively low specific activity and the small fraction of radioactive atoms of ⁸⁹Sr in the administered dose has to compete for binding sites in bone with a much greater fraction of atoms of non-radioactive ⁸⁸Sr.

Consequently there is the need for more effective treatment of bone metastases and other skeletal tumors for improved palliative effect and potential destruction of disseminated metastases.

### OBJECTS OF THE INVENTION

An object of the present invention is to provide a method for more efficacious treatment of bone metastases and other skeletal tumors.

Another object of the present invention is to provide a method for substantially reducing pain intensity resulting from tumor growth.

A still further object of the present invention is to provide for a method having improved destructive capability.

Yet another object of the present invention was to provide a method for more effectively quantifying the size of malignant tumor growth.

A still further object of the present invention is to permit more quantitative treatment of bone metastases.

A still further object of the present invention is to provide a method and concomitant palliative effect.

A still further object of the present invention is to provide a composition of matter substantially more effective in the destruction of a cancer such as bone metastases with improved palliative effect and destruction capabilities.

### SUMMARY OF THE INVENTION

These and other objects of the present invention are achieved by administering a physiological acceptable amount of a carrier free strontium radioisotope to an individual having cancer such as bone metastases.

### DETAILED DESCRIPTION OF THE INVENTION

Carrier-free ⁸⁹Sr is produced from 89Y and is obtained with a specific activity of 17 Ci/mg of strontium, which means that 1 mCi contains only 58.8 ng of strontium, where all atoms are radioactive. Until very recently the available ⁸⁹Sr was produced from ⁸⁸Sr at a specific activity on average of 120 μCi/mg of strontium, which means that 1 mCi contains 8.333 mg of strontium, where only 58.8 nanograms are of radioactive strontium (⁸⁹Sr). Therefore, a standard dose of 4 mCi (148 MBq) would contain a total amount of 33.332 mg of strontium, of which only 235.2 ng would correspond to radioactive ⁸⁹Sr. This means that each nanogram of the radioactive species needs to compete with 141 micrograms of the non-radioactive variety for the same organic binding sites. Therefore, one atom of radioactive ⁸⁹Sr needs to compete against 141,000 atoms of non-radioactive ⁸⁸Sr. As a consequence, the fractional uptake of low specific activity ⁸⁹Sr by bone metastases is relatively low and slow as compared to the uptake obtained by using carrier-free ⁸⁹Sr.

TABLE I
Proportion of radioactive ⁸⁹Sr and total strontium in new carrier-free
⁸⁹Sr chloride (17 Ci/mg Sr) and in commercially available
⁸⁹Sr chloride (120 μCi/mg Sr).

| | Carrier-free | | | | | |
|---|---|---|---|---|---|---|
| | (17 Ci/mg Sr) | | | (120 μCi/mg Sr) | | |
| | 1 μCi | 1 mCi | 1 Ci | 1 μCi | 1 mCi | 1 Ci |
| Radio- | 58.8 pg | 58.8 ng | 58.8 μg | 58.8 pg | 58.8 ng | 58.8 μg |
| active ⁸⁹Sr | | | | | | |
| Total Strontium | 58.8 pg | 58.8 ng | 58.8 μg | 8.3 μg | 8.3 mg | 8.3 g |

| | | |
|---|---|---|
| Strontium/⁸⁹Sr | 58.8 ng/58.8 ng = 1.0 | 58.8 ng/8.3 mg = |
| | 7,506 × 10⁻⁶ | |

The fractional uptake (% of the dose) of ⁸⁹Sr by bone metastases and the renal clearance of radioactive strontium would be enhanced in proportion to the increase in specific activity, proportionally reducing the fraction of the radionuclide remaining in blood, which is available to normal bone. The results would be a significant enhancement of the therapeutic index (target/non-target ratio) of ⁸⁹Sr in palliation of painful metastases and a reduction of its toxic effects on bone marrow.

⁸⁹Sr can be produced in a fast nuclear reactor by bombardment of stable yttrium-89 with a neutron flux of up to 3.7×10¹⁵ neutrons/m²/second. The reaction involved is ⁸⁹Y(n, p), according to the equation:

⁸⁹Y+neutron→⁸⁹Sr+proton

The result of this reaction is the formation of a different element (⁸⁹Sr) than the target element (89Y) So, the target (yttrium) and the product (strontium) can be separated by chemical methods, resulting in carrier-free ⁸⁹Sr with specific activity of 17 Ci (629 GBq)/mg of strontium, which is the highest possible specific activity for this element, and where all atoms of strontium are of the same radioisotope (⁸⁹Sr). The specific activity remains the same independent of the radioactive decay of the radionuclide.

Several lists of therapeutic radionuclides have been generated over the years but ⁸⁹Sr is not included in them. A number of basic properties of these nuclides are common to all of them. Strontium-89 shares all these characteristics with therapeutic radionuclides but one. All therapeutic radionuclides are of carrier-free quality, but commercially available ⁸⁹Sr is of a very low specific activity.

One embodiment of the present invention is a method to calculate the activity of the dose of carrier-free ⁸⁹Sr chloride (17 Ci [628 GBq]/mg of strontium) which would deliver the same radiation dose to bone metastasis as the provided by the customary dose of 4.0 mCi (148 MBq) of ⁸⁹Sr chloride at standard specific activity [120 μCi [4.44 MBq]/mg of strontium] for palliation of painful bone metastasis.

Another embodiment of therapeutic invention is a method to calculate patient-specific doses made of carrier-free ⁸⁹Sr chloride on the basis of osteoblastic activity of the individual metastatic burden, estimated by quantitative imaging with a gamma camera of a tracer dose of ⁸⁵Sr chloride.

In both cases, provision is made for monitoring the distribution of the administered carrier-free ⁸⁹Sr salt in the patient by adding to the dose tracer amounts of a carrier-free salt of ⁸⁵Sr for imaging and quantitation with a gamma camera.

Another embodiment is the application of a salt of carrier-free ⁸⁹Sr, preferably chloride, (17 Ci [628 GBq]/mg of strontium) in the treatment and imaging of bone metastases and other osteoblastic skeletal tumors. As in the previous embodiments, the basis is the enhancement of the fractional uptake of ⁸⁹Sr chloride by bone metastases and by osteoblastic skeletal tumors by the increased specific activity of the radiopharmaceutical from 120 μCi [4.44 MBq]/mg of strontium to 17 Ci [628 GBq]/mg of strontium. However, the aim is not to palliate painful metastases but to deliver a tumoricidal absorbed radiation dose for their eradication.

The range of the radiation absorbed dose to deliver to bone metastases must be decided upon with knowledge and experience in the art and oncological criteria. But the activity of the dose expected to deliver the selected range of absorbed radiation dose would be decided upon by measuring the activities in the metastasis after the administration of 1 mCi (37 MBq) of ⁸⁵Sr chloride at standard specific activity (120 μCi (4.44 MBq)/mg of strontium), as described in planning the dose of carrier-free ⁸⁹Sr chloride for palliation of pain in bone metastasis.

As hereinabove discussed, provision may be made for monitoring the distribution of the administered carrier-free ⁸⁹Sr salt in the patient by adding dose tracer amounts of a carrier-free salt of ⁸⁵Sr for imaging and quantitation with a gamma camera. If the absorbed radiation dose is too high, or if the patient has multiple widespread metastasis in skeleton (which may cause severe toxic effects on bone marrow and myelosuppression), the dose may be fractionated into two or more smaller doses.

To calculate the activity (mCi or MBq) of the dose of a carrier-free salt of ⁸⁹Sr, preferably chloride, to be administered to palliate painful metastases in a patient, 1 mCi (37 MBq) of ⁸⁵Sr chloride with standard specific activity [120 μCi (4.44 MBq)/mg of strontium) is injected into the patient and radioactivity in each metastasis is measured 24 hours after injection. The fractional uptake in each metastasis (relative osteoblastic activity) is calculated by expressing the radioactivity measured in each metastasis as a percent of the administered dose. The sum of the fractional uptake in all metastases is taken as the “metastatic burden” of the patient.

Measurements of radioactivity can be guided by a recent bone scan with ⁹⁹Tc^{m}-MDP (methylene diphosphonate). These measurements can be made by external point counting with a collimated scintillation detector, or by using a gamma camera. In both cases, the pulse height analyzer should be set with the window centered at 514 keV, and the sensitivity of the counting system for the gamma photons emitted by ⁸⁵Sr (counts/second/mCi or MBq) need to be verified before measurements. However, the use of a gamma camera provides a more objective and precise information than the obtained by external point counting.

The gamma camera needs to be equipped with a high-energy collimator. Anterior and posterior images of the lesions are recorded for 15 minutes. The opposing scintgraphic images of a given lesion are aligned by cross-correlating the x- and y- projections of the images. An irregular region of interest (ROI) is drawn around each lesion, and this is superimposed on all the images of a given lesion to obtain the anterior and posterior count rates (counts/second).

The count rate in each metastasis is calculated by the square root of the product of the count rates in the anterior (A) and posterior (P) images divided by e^{-μd}: [(cps A) ×(cps P)/(e^{-μd})]. The exponential term accounts for the attenuation of the gamma photons by the patient's tissues, where μ is the attenuation coefficient for the gamma rays of ⁸⁵Sr, previously measured in a water phantom, and d is the patent thickness measured in cm from x-ray images. The attenuation coefficient needs to be experimentally estimated only once; sensitivity should be measured using an ⁸⁵Sr source in a soft-tissue phantom before each imaging session because it varies with time. The count rate in each metastasis, corrected by attenuation, (counts/second), is divided by the sensitivity of the gamma camera (counts per second/mCi or MBq) to obtain the activity in the lesions in mCi or MBq:

(cps in metastasis)/(cps/mCi of ⁸⁵Sr)=mCi

The result is multiplied by 100 and divided by the injected activity in mCi or MBq to estimate the fractional uptake (%) by each metastasis:

(mCi in metastasis)×100/Injected activity (mCi)=fractional uptake (%)

The use of high-energy gamma-emitting radionuclides such as ⁸⁵Sr results in images of relatively poor quality because the sensitivity of the gamma camera for energetic radiation is very low, due to the low geometric efficiency of the high-energy collimator and the low sensitivity of the detecting crystal. The spatial resolution of the gamma camera is also poor because the thick septa in the high-energy collimator. If two or more metastases are closely spaced the activity would be quantified by using a single ROI around them.

Notwithstanding the inaccuracies in the measurement of the fractional uptake of ⁸⁵Sr chloride by the metastases, this method of calculation of the activity to be administered to a patient is more precise than the use of a fixed dose or a dose calculated on the basis of body weight. The biological distribution of ⁸⁵Sr chloride with standard specific activity [120 μCi (4.44 MBq)/mg of Sr] is identical to the biological distribution of ⁸⁹Sr chloride of the same specific activity. In other words, the scintigraphic images and the measurements of radioactivity after the administration of ⁸⁵Sr chloride are a good representation of the actual distribution of ⁸⁹Sr of the same specific activity. These images allow quantitation of the number of metastases and their relative osteoblastic activity.

The sum of the fractional uptake of ⁸⁵Sr chloride by all detectable metastases (“metastatic burden”), is used to define the activity (mCi or MBq) of ⁸⁹Sr chloride needed to palliate painful metastases in a patient. A study performed with 1 mCi of ⁸⁵Sr chloride with standard specific activity [120 μCi (4.44 MBq)/mg of strontium] in a patient with painful bone metastases from prostate cancer depicted 5 well defined areas of increased uptake in the skeleton, coinciding with bone metastases evident in the bone scan previously performed with ⁹⁹Tc^{m}-MDP as well as with painful sites. The fractional uptake measured in these metastases was 3%, 4%, 7%, 10%, and 17% of the dose respectively, with a total “metastatic burden” of 41% (3+4+7+10+17=41) (Table II). The objective is to administer a dose of carrier-free ⁸⁹Sr chloride that would deliver a similar absorbed radiation dose in the metastasis as a standard dose of 4 mCi at standard specific activity.

The fractional uptake (%) of a standard dose ⁸⁹Sr chloride for pain palliation in bone metastasis [4 mCi (148 MBq)] with specific activity of 120 μCi (4.44 MBq)/mg of strontium) (B) is calculated from the fractional uptake (%) of 1 mCi of ⁸⁵Sr chloride at the same specific activity (A) is illustrated in Table II.

TABLE II

| | |
|---|---|
| A | B |
| 1 mCi of ⁸⁵Sr Chloride | 4 mCi of ⁸⁹Sr Chloride |
| (120 μCi/mg Sr) | (120 μCi/mg Sr) |

| | Uptake | Uptake | Uptake | Uptake | Uptake | Uptake |
|---|---|---|---|---|---|---|
| Metastasis | (%) | (mg) | (μCi) | (%) | (mg) | (μCi) |
| 1 | 3.0 | 0.250 | 30 | 3.0 | 1.000 | 120 |
| 2 | 4.0 | 0.333 | 40 | 4.0 | 1.332 | 160 |
| 3 | 7.0 | 0.583 | 70 | 7.0 | 2.332 | 280 |
| 4 | 10.0 | 0.833 | 100 | 10.0 | 3.332 | 400 |
| 5 | 17.0 | 1.417 | 170 | 17.0 | 5.668 | 680 |
| Total | 41.0 | 3.416 | 410 | 41.0 | 13.664 | 1,640 |

The fractional uptake (%) of a standard dose of 4.0 mCi (148 MBq) of ⁸⁹Sr chloride at the standard specific activity is calculated for each metastasis from the results obtained with ⁸⁵Sr chloride. Since the specific activity is the same for both radionuclides, the fractional uptake would be similar if the same physiological conditions are maintained. However, the activity (μCi) and the amount of strontium (mg) incorporated by the metastases would be increased by four times because the activity of the administered dose is increased from 1 mCi to 4 mCi. Theoretically, the fractional uptake of a standard dose of ⁸⁹Sr chloride by the metastatic burden would be 41.0% of the dose (1.64 mCi or 60.7 MBq), leaving in blood a remaining fraction of 59% of the dose (2.36 mCi or 87.3 MBq) available for renal excretion and for incorporation by normal bone.

The absolute amount of strontium (mg) incorporated by each lesion can be calculated by multiplying the activity of the administered dose (mCi or MBq) by the fractional uptake by a given metastasis (% of the dose), and dividing the result by the specific activity of the dose (mCi or MBq/mg of strontium):

Strontium (mg)=Activity of the dose (mCi) × fractional uptake (%)/specific activity (mCi/mg Sr)

In the case of the metastasis No. 1, presenting a fractional uptake of 3%:

4.0 mCi ×0.03=0.120 mCi=120 μCi;

120 μCi/120 μCi per mg=1.0 mg of strontium.

The other metastasis would incorporate 1.332 mg, 2.332 mg, 3.332 mg, and 5.668 mg respectively, with a total of 13.664 mg of strontium incorporated by all metastases:

4.0 mCi ×0.41 =1.64 mCi =1,640 μCi;

1,640 μCi/120. μCi per mg=13.664 mg of strontium.

In Table II it is also appreciated that the fractional uptake is the same for both the activity (μCi) and the absolute amount (mg) of strontium incorporated by the metastases. One mg of strontium corresponds to 3% of the element in the dose. One hundred and twenty μCi of ⁸⁹Sr correspond to 3% of the activity in the dose. One mCi of strontium chloride with specific activity of 120 μCi/mg of strontium contains 8.333 mg of strontium (Table I), therefore, 4 mCi would contain 33.333 mg of the element. A fractional uptake of 41.0% corresponds to 410 μCi and 3.416 mg when using 1 mCi, and to 1.64 mCi and 13.667 mg when the dose is increased to 4 mCi (Table II). The amount of strontium (mg) taken up by the metastasis per unit time should be constant if the physiological parameters are maintained. Dose calculation may be guided by the relationship of the amount of strontium in carrier-free ⁸⁹Sr chloride (17 mCi/mg of Sr) and that which is in an equal amount of its low specific activity counterpart (120 μCi/mg of Sr). The amount of strontium in 4 mCi of low specific activity ⁸⁹Sr chloride (120 μCi/mg of Sr), is 33.333 mg, while in carrier-free ⁸9Sr chloride (17 Ci/mg of Sr) it is reduced to 235.2 ng. The relationship between these quantities would be:

235.2 ng/33.333 mg=7.056×10−6

Theoretical distribution of ⁸⁹Sr chloride at different specific activities assuming the same fractional uptake (% of the dose) by the metastases. (A) ⁸⁹Sr chloride with low specific activity [120 μCi (4.44 MBq)/mg of strontium]; (B) carrier-free ⁸⁹Sr chloride [17 Ci (629 GBq)/mg of strontium] is illustrated in Table III.

TABLE III

| | |
|---|---|
| A | B |
| 4 mCi of ⁸⁹Sr Chloride | 4 mCi of ⁸⁹Sr Chloride |
| (120 μCi/mg Sr) | (17 Ci/mg Sr) |

| | Uptake | Uptake | Uptake | Uptake | Uptake | Uptake |
|---|---|---|---|---|---|---|
| Metastasis | (%) | (mg) | (Ci) | (%) | (ng) | (Ci) |
| 1 | 3.0 | 1.000 | 120 | 3.0 | 7.056 | 120 |
| 2 | 4.0 | 1.332 | 160 | 4.0 | 9.400 | 160 |
| 3 | 7.0 | 2.332 | 280 | 7.0 | 16.456 | 280 |
| 4 | 10.0 | 3.332 | 400 | 10.0 | 23.513 | 400 |
| 5 | 17.0 | 5.668 | 680 | 17.0 | 39.997 | 680 |
| Total | 41.0 | 13.664 | 1,640 | 41.0 | 96.422 | 1,640 |

If the “metastatic burden” takes up 41% of a 4mCi dose of the low specific activity ⁸⁹Sr chloride (13.664 mg of strontium) (Tables II and III), it is evident that the very small amount of strontium in the carrier-free ⁸⁹Sr chloride dose (235.2 ng) would be totally incorporated by the metastases, resulting in a fractional uptake of 100% y the metastatic burden. To correct for this effect, the data in Table IlI should be multiplied by the ratio between 100% uptake and the fractional uptake by the metastatic burden by using 89Sr chloride at the lower specific activity. In this case, the ratio is 100%/41%=2.439.

Table IV-A below shows the theoretical fractional uptake of a dose of 4 mCi (148 MBq) of carrier-free ⁸⁹Sr chloride by the metastases, which is 2.439 times larger than the fractional uptake of the same activity of 89Sr chloride of standard specific activity. Consequently, for the metastases to receive a similar radiation dose than with 4.0 mCi (148 MBq) of ⁸⁹Sr chloride at standard specific activity, the activity of the dose needs to be reduced proportionally to 1.64 mCi (60.68 MBq), because 4.00 mCi/2.439=1.64, and 148 MBq/2.439=60.68 MBq is shown in Table IV-B.

TABLE IV

| | |
|---|---|
| A | B |
| 4.0 mCi of ⁸⁹Sr Chloride | 1.64 mCi of ⁸⁹Sr Chloride |
| (17 Ci/mg Sr) | (17 Ci/mg Sr) |

| | Uptake | Uptake | Uptake | Uptake | Uptake | Uptake |
|---|---|---|---|---|---|---|
| Metastasis | (%) | (ng) | (μCi) | (%) | (ng) | (μCi) |
| 1 | 7.3 | 17.209 | 292 | 7.3 | 7.056 | 120 |
| 2 | 9.8 | 22.927 | 392 | 9.8 | 9.400 | 160 |
| 3 | 17.0 | 40.136 | 680 | 17.0 | 16.456 | 280 |
| 4 | 24.4 | 57.348 | 976 | 24.4 | 23.513 | 400 |
| 5 | 41.5 | 97.553 | 1666 | 41.5 | 39.997 | 680 |
| Total | 100.0 | 235.173 | 4000 | 100.0 | 96.422 | 1640 |

Table V compares the distribution of a standard dose [4.0 mCi (148 MBq)] of ⁸9Sr chloride at low secific activity (120 μCi/mg of Sr) with the distribution of the same activity of carrier-free 8⁹Sr chloride (17 Ci/mg of Sr).

TABLE V

| | |
|---|---|
| A | B |
| 4.0 mCi of ⁸⁹Sr Chloride | 1.64 mCi of ⁸⁹Sr Chloride |
| (120 μCi/mg Sr) | (17 Ci/mg Sr) |

| | Uptake | Uptake | Uptake | Uptake | Uptake | Uptake |
|---|---|---|---|---|---|---|
| Metastasis | (%) | (mg) | (μCi) | (%) | (ng) | (μCi) |
| 1 | 3.0 | 1.000 | 120 | 7.3 | 7.056 | 120 |
| 2 | 4.0 | 1.332 | 160 | 9.8 | 9.400 | 160 |
| 3 | 7.0 | 2.332 | 280 | 17.0 | 16.456 | 280 |
| 4 | 10.0 | 3.332 | 400 | 24.4 | 23.513 | 400 |
| 5 | 17.0 | 5.668 | 680 | 41.5 | 39.997 | 680 |
| Total | 41.0 | 13.664 | 1,640 | 100.0 | 96.422 | 1,640 |

If a patient is administered 1.64 mCi of carrier-free ⁸⁹Sr chloride, the fractional uptake (%) by the metastatic burden would increase from 41% to 100%, enhancing the ratio metastases to normal bone 2.439 times from the measured 8-40 to theoretical values of 20-98. Although the absolute amount of strontium (mg) is reduced from 13.664 mg to only 96.422 ng, the radiation dose delivered to the metastases, represented by the fractional uptake in μCi, would be similar to that attained when administering a standard dose of 4.0 mCi (148 MBq) of ⁸⁹Sr chloride with specific activity of 120 μCi/mg of strontium.

The dose of carrier-free ⁸⁹Sr chloride to be administered to the patient could be added to with 200-400 μCi (7.4-14.8 MBq) of carrier-free ⁸⁵Sr chloride so as to corroborate the distribution of radiostrontium in the patient's body with a gamma camera after the administration of the dose.

The method herein described offer the following advantages over the administration of 4 mCi (148 MBq) of ⁸⁹Sr chloride at standard specific activity (120 μCi/mg of strontium):

1. It reduces the whole body irradiation of the patient by enabling the use of a smaller activity of ⁸⁹Sr;

2. It reduces and may prevent the pharmacological effect of ⁸⁹Sr chloride (facial flushing during its injection) by reducing its chemical amount in the dose from mg to ng;

3. It reduces bone marrow irradiation and myelosuppression by the reduction of the activity in the dose of ⁸⁹Sr;

4. It further reduces bone marrow irradiation and myelosuppression by greatly diminishing the uptake of ⁸⁹Sr by normal bone;

5. It increases the therapeutic index (metastases to normal bone ratio) of ⁸⁹Sr chloride.

6. The palliative effect would be the same as the previous art because a similar amount of radioactivity would be taken up by the metastases.

7. The actual distribution of the administered dose of ⁸⁹Sr chloride can be conveniently monitored with a gamma camera by detecting the gamma photons emitted by the added carrier-free ⁸⁵Sr chloride.

The method, described hereinabove provides a method to replace a dose of 4.0 mCi (148 MBq) of ⁸⁹Sr chloride at standard specific activity (120 μCi/mg of strontium) with a smaller dose of carrier-free ⁸⁹Sr chloride (17 Ci/mg of strontium), but does not allow the calculation of patient-specific dose in relation to the osteoblastic activity of the metastatic burden. In order to do so, it would is necessary to use the previously described method in a sufficient number of patients to collect quantitative information on the range of radioactivity (μCi) needed in a given metastasis to palliate pain. Once this information is available, it is possible to calculate the patient-specific dose by measuring the fractional uptake of a small dose of carrier-free ⁸⁵Sr chloride by each metastasis.

Thus, if the only painful metastases are metastasis No. 4 and No. 5 (Table V), the calculated 1.64 mCi (60.68 MBq) of carrier-free ⁸⁹Sr chloride would suffice to palliate pain, since these metastases are incorporating 400 μCi (14.8 MBq) and 680 μCi (25.2 MBq), respectively, values which are inside the proposed range [300 μCi (11.1 MBq) to 700 μCi (25.9 MBq)].

However, if all of the five metastases are painful, then 1.64 mCi (60.68 MBq) of carrier-free ⁸⁹Sr chloride would not be enough to palliate pain, since metastasis No. 1, No. 2, and No. 3 would be taking up only 120 μCi (4.44 MBq), 160 μCi (5.92 MBq) and 280 μCi (10.36 MBq), respectively. Such amounts are below the lower limit of the palliative range so such metastases would continue producing pain after administration of the radiopharmaceutical. Therefore, the activity in the dose should be incremented to include all metastases in the proper absorbed radiation dose range.

The ratio between the lower limit of the palliative range and the activity taken up by the less active metastasis is used in the calculation of the final dose. In this case, 300 μCi/120 μCi=2.5, i.e., the activity in the dose should be increased by 2.5 times to 4.1 mCi (1.64 mCi×2.5=4.1 mCi) (Table VI). Since the fractional uptake (%) is the same, independent of the total activity of the dose, the metastasis would receive 300 μCi (11.1 MBq), 400 μCi (14.8 MBq), 700 μCi (25.9 MBq), 1,000 μCi (37.0 MBq), and 1,700 μCi (62.9 MBq), respectively (as set forth in Table VI), increasing the probability of total pain relief. However, the toxic effects of this dose on bone marrow would be limited to those areas within the vicinity of the metastases. Areas surround by normal bone would not be irradiated because of the increased target to non-target ratio.

TABLE VI

| | |
|---|---|
| A | B |
| 1.64 mCi of Carrier-free ⁸⁹Sr | 4.1 mCi of Carrier-free ⁸⁹Sr |
| Chloride (17 Ci/mg Sr) | Chloride (17 Ci/mg Sr) |

| | Uptake | Uptake | Uptake | Uptake | Uptake | Uptake |
|---|---|---|---|---|---|---|
| Metastasis | (%) | (ng) | (μCi) | (%) | (ng) | (μCi) |
| 1 | 7.3 | 7.056 | 120 | 7.3 | 17.640 | 300 |
| 2 | 9.8 | 9.400 | 160 | 9.8 | 23.500 | 400 |
| 3 | 17.0 | 16.456 | 280 | 17.0 | 41.140 | 700 |
| 4 | 24.4 | 23.513 | 400 | 24.4 | 58.783 | 1,000 |
| 5 | 41.5 | 39.997 | 680 | 41.5 | 99.992 | 1,700 |
| Total | 100.0 | 96.442 | 1,640 | 100.0 | 241.055 | 4,100 |

As with the customary administration of 4 mCi (148 MBq) of ⁸⁹Sr chloride at standard specific activity (120 μCi/mg of Sr), carrier-free ⁸⁹Sr chloride administration would be repeated every three months, previous estimation of the fractional uptake of carrier-free ⁸⁵Sr chloride, as described. It could also be administered more frequently if signs of myelosuppression are not evident in weekly blood counts.

The biochemically targeting of radiotherapy of malignant bone metastases and other osteoblastic skeletal tumors with a carrier free salt of ⁸⁹Sr, preferably chloride, which until now has been prevented by the toxic effects on bone marrow partially produced by the uptake of the radionuclide by normal bone, and by the consequent relatively low therapeutic index (target/non-target ratio) of ⁸⁹Sr chloride at the standard specific activity (120 μCi (4.44 MBq)/mg of strontum). This limitation vanished with the availability of carrier-free ⁸⁹Sr chloride with specific activity of 17 mCi (629 GBq)/mg of strontium.

As hereinabove discussed, the basis of this embodiment is the enhancement of the fractional uptake of ⁸⁹Sr chloride by bone metastases and by osteoblastic skeletal tumors as a result of the increased specific activity of the radioactive salt from 120 μCi (4.44 MBq)/mg of strontium to 17 Ci (629 GBq)/mg of strontium. However, the aim in this case is not to palliate painful metastases but to deliver a tumoricidal absorbed radiation dose for eradication of the lesions.

The ability of the individual metastases and other osteoblastic skeletal tumors to take up and retain radiostrontium, is the most important factor in determining the absorbed radiation dose delivered to the metastases. The absorbed radiation dose is variable from lesion to lesion because the inhomogeneous nature of ⁸⁹Sr deposition in different metastatic lesions, each with a particular concentration rate. This means that a single dose of ⁸⁹Sr chloride would not deliver the same absorbed radiation dose to all metastases in a patient. This is illustrated in Table II by the variation of the fractional uptake from one metastasis to another. The mean fractional uptake is 8.2%, with a range from 3% to 17%. Therefore, each lesion would be delivered with a different absorbed radiation dose as measured in grays (Gy).

Absorbed radiation dose estimates for metastatic lesions in bone, obtained by direct measurements of radioactivity in bone metastases after the administration of 4 mCi (148 MBq) of ⁸⁹Sr chloride at standard specific activity (120 μCi [4.44 MBq]/mg of strontium), varied from 1.3 to 64 Gy, with a mean of 18±16 Gy. The average total absorbed dose in normal bone sites was 1.1±0.4 Gy. The metastases (target) to normal bone (non-target) dose ratio in individual samples varied from 8±4 to 40±25. If compared to absorbed radiation dose provided by external beam radiotherapy, the absorbed radiation dose delivered by ⁸⁹Sr chloride should be large enough to produce a therapeutic effect on the metastases. But absorbed radiation doses delivered by ⁸⁹Sr chloride may not be compared with those delivered by external beam radiotherapy because the different dose rates used in each methodology. External beam radiotherapy delivers very high dose rates in a very short time, while ⁸⁹Sr chloride provides very low dose rates during a long period. These differences reduce the biological effect of a given dose of ⁸⁹Sr because there is repair of sublethal damage. An analogy can be drawn from the fact that 60 to 70 Gy are highly effective in eradicating a small volume prostate cancer when delivered by external beam radiotherapy, but when using a permanent ¹²⁵I implant (with similar physical half-life and dose rates to those of ⁸⁹Sr) the required dose is in the range of 160 to 240 Gy (2.3 to 4.0 times higher).

The range of radiation absorbed doses to deliver to bone metastases must be decided upon with knowledge and experience in the art and oncological criteria. But the activity of the dose expected to deliver the selected range of absorbed radiation dose would be decided by measuring the activities in the metastasis after the administration of 1 mCi (37 MBq) of ⁸⁵Sr chloride at standard specific activity (120 μCi (4.44 MBq)/mg of strontium), as described in planning the dose of carrier-free ⁸⁹Sr chloride for palliation of pain in bone metastasis in the previous preferred embodiment.

To provide a therapeutic dose of carrier-free ⁸⁹Sr chloride to eradicate the metastasis, instead of just a pain palliative dose, it is necessary to deliver a mean absorbed radiation dose of 60 Gy to the metastatic lesions.

As in Example 1, the study performed with ⁸⁵Sr chloride [specific activity of 120 μCi (4.44 MBq)/mg of strontium] depicts five well defined areas of increased uptake in the skeleton, coinciding with bone metastases evident in the skeletal scan performed with 99Tc^{m}-MDP as well as with painful sites. The fractional uptake measured in these metastases was 3%. 4%, 7%, 10%, and 17% of the dose, respectively, with a total “metastatic burden” of 41% (3+4+7+10+17=41) (Table II).

It is calculated that a standard dose of ⁸⁹Sr chloride [4.0 mCi (148MBq); 120 μCi (4.44 MBq)/mg of strontium] would incorporate similar fractional amounts of the administered dose in the metastases [41% =1.64 mCi (60.68 MBq)] (Table 11), leaving in blood a fraction of 59% [2.36 mCi (87.32 MBq)] available for renal excretion and for incorporation into normal bone. It is further assumed that the dose would deliver a mean absorbed radiation dose of 18±16 Gy to the metastatic lesions.

Since the mean absorbed radiation dose needs to be increased from 18 Gy to 60 Gy, the activity in the metastasis have to be increased 3.333 times (60/18=3.333). This could be achieved by increasing the activity of the dose from 4.0 mCi (148 MBq) to 13.332 mCi (493.28 MBq). However, in this case, the fraction remaining in blood, available for renal clearance and uptake by normal bone, would increase from 2.36 mCi (87.32 MBq) to 7.866 mCi (291 MBq), with serious toxic effects on bone marrow and a high whole-body irradiation. Besides, such a high dose may also increase potentially undesired pharmacological effects of strontium chloride (facial flushing during injection). But the use of carrier-free ⁸⁹Sr chloride, as in the previous preferred embodiment, would enhance the fractional uptake by the metastases. Table V shows that 1.64 mCi (60.68 MBq) of carrier-free ⁸⁹Sr chloride delivers the same amount of radioactivity (μCi) to the metastases than 4.0 mCi (148 MBq) of standard ⁸⁹Sr chloride. This means that a dose of carrier-free ⁸⁹Sr chloride 3.333 times more active [1.64 mCi (60.7 MBq) ×3.333=5.466 mCi (202.2 MBq)] would deliver a mean absorbed radiation dose of 60 Gy to the metastases (illustrated in Table VIl), while strictly limiting the fraction of the radionuclide available to normal bone.

TABLE VII

| | |
|---|---|
| A | B |
| 13.332 mCi of ⁸⁹Sr | 5.466 mCi |
| Chloride (120 μCi/mg Sr) | of Carrier-free ⁸⁹Sr |

| | Uptake | Uptake | Uptake | Uptake | Uptake | Uptake |
|---|---|---|---|---|---|---|
| Metastasis | (%) | (mg) | (μCi) | (%) | (ng) | (μCi) |
| 1 | 3.0 | 3.333 | 400 | 7.3 | 23.518 | 400 |
| 2 | 4.0 | 4.440 | 533 | 9.8 | 31.330 | 533 |
| 3 | 7.0 | 7.723 | 933 | 17.0 | 54.848 | 933 |
| 4 | 10.0 | 11.106 | 1,332 | 24.4 | 78.369 | 1,332 |
| 5 | 17.0 | 18.891 | 2,266 | 41.5 | 133.310 | 2,266 |
| Total | 41.0 | 45.493 | 5,466 | 100.0 | 321.375 | 5,466 |

Even though the uptake by normal bone is greatly decreased, it is still recommended to monitor blood counts once a week to detect any myelosuppressive effects, which may be caused by the augmented irradiation of bone marrow in the vicinity of the bone metastases.

The dose of ⁸⁹Sr chloride could be added with 0.5-1.0 mCi of carrier-free ⁸⁵Sr chloride to verify with a gamma camera that the biodistribution of the radiopharmaceutical resulted appropriate.

If the prescribed mean absorbed radiation dose is higher than 60 Gy and the oncologist or nuclear physician believes it to be excessive, or when the patient has multiple widespread metastases in skeleton which may cause severe toxic effects on bone marrow and myelosuppression, the prescribed dose may be fractionated in two or more smaller doses.

While specific embodiments of the present invention have been shown and described to illustrate the inventive principles, it is to be understood that such a description have been offered only by way of example and not by way of limitation, it is therefore manifestly intended that the invention be limited only by the claims and the equivalents thereof.

What is claimed:

1. A method for treating bone metastases and like skeletal tumors in an individual which comprises treating said individual with a dose of a physiologically acceptable amount of carrier-free radioactive strontum-89 chloride.

2. A method for treating bone metastases and like skeletal tumors as defined in claim 1 wherein activity of said dose of carrier-free radioactive strontium-89 chloride is 17 Ci (628 GBq)/mg of strontium.

3. The method for treating bone metastases and like skeletal tumors in accordance with claim 1 wherein said dose of said carrier-free radioactive strontium-89 chloride is established by quantitative imaging with a tracer dose of strontum-85 chloride administered to said individual.

4. The method for treating bone metastases and like skeletal tumors in accordance with claim 1 wherein said carrier-free radioactive strontium-89 is produced by neutron bombardment of ytttrium-89.

5. A method for preparing carrier-free radioactive strontium-89 which comprises:
Subjecting yttrium-89 to neutron bombardment to form strontium-89; and
Separating strontium-89 from unconverted yttrium-89.
